(19) United States
(12) Patent Application Publication
(10) Pub. No.: US 2021/0113145 A1
(43) Pub. Date: Apr. 22, 2021

(54) LOW COST, TRANSFERRABLE AND THERMALLY STABLE SENSOR ARRAY PATTERNED ON CONDUCTIVE SUBSTRATE FOR BIOFLUID ANALYSIS

(71) Applicant: THE REGENTS OF THE UNIVERSITY OF CALIFORNIA, Oakland, CA (US)

(72) Inventors: Sam EMAMINEJAD, Los Angeles, CA (US); Yichao ZHAO, Los Angeles, CA (US)

(73) Assignee: THE REGENTS OF THE UNIVERSITY OF CALIFORNIA, Oakland, CA (US)

(21) Appl. No.: 17/048,548

(22) PCT Filed: Apr. 18, 2019
(86) PCT No.: PCT/US2019/028054

§ 371 Date: Oct. 16, 2020

Related U.S. Application Data
(60) Provisional application No. 62/660,173, filed on Apr. 19, 2018.

(51) Int. Cl. A61B 5/00; A61B 5/259; A61B 5/145; A61B 5/1495

(52) U.S. Cl. CPC A61B 5/4266; A61B 5/259; A61B 2562/046; A61B 5/1495; A61B 5/14532

(57) ABSTRACT

A disposable sensor for biofluid analysis includes: (1) a conductive film having a first major surface and a second major surface opposite to the first major surface; (2) a sensing layer disposed on the first major surface of the conductive film; and (3) an adhesive layer disposed on the second major surface of the conductive film.

### CROSS-REFERENCE TO RELATED APPLICATIONS

This application claims the benefit of U.S. Provisional Application No. 62/660,173, filed Apr. 19, 2018, the contents of which are incorporated herein by reference in their entirety.

### TECHNICAL FIELD

This disclosure generally relates to a sensor, a sensor array, and a method for biofluid analysis.

### BACKGROUND

Recent advances in electrochemical sensor development, flexible device fabrication and integration technologies, and low-power electronics have prompted the development of wearable sweat sensors. Some wearable sweat sensors have demonstrated the in-situ sensing of various sweat analytes. However, such sensors lacked the ability to induce sweat on-demand and periodic analysis. The inaccessibility of sweat in sedentary individuals and lack of control of the secretion process impede the exploitation of the benefits associated with the non-invasive modality of sweat analysis. Also, further challenges remain in order to exploit sweat analysis for continuous health monitoring. In particular, a cost of a disposable sensing module should be substantially lowered, and a sensing layer's functionality should be preserved for extended operation at about room temperature to realize frequent sample analysis in uncontrolled environments (e.g., on-body testing).

It is against this background that a need arose to develop the embodiments described herein.

### SUMMARY

Some embodiments are directed to a low cost, thermally stable, disposable sensor array which is patterned on a conductive, adhesive substrate and hence can be readily adhered onto permanent electrode contacts integrated within a wearable device including electronic readout and control functionality. This methodology provides a cost-effective solution for wearable and mobile biofluid analysis platforms, such as for analysis of saliva, urine, interstitial fluid, and sweat, which specify frequent sample analysis using a fresh/uncontaminated sensing interface.

A comparison design for biofluid analysis typically includes a disposable sensing module (including an electrochemical sensor array along with associated electrode contacts and electrical interconnects that are disposed on a common substrate), which in turn interfaces with a permanent circuit board providing control, signal processing and wireless transmission functionality. The sensing module is realized via direct formation of electrochemical sensing layers on pre-fabricated/printed electrode contacts. Therefore, with the comparison design, the electrode contacts and associated electrical interconnects are discarded along with the sensing layers after a sensing operation, since effectively they are incorporated in the same substrate and therefore cannot be readily refreshed for subsequent analysis. Moreover, a poor thermal stability of some sensors impedes their practical use in applications where biofluid sample analysis for an extended amount of time in uncontrolled environment is desired.

Here, in some embodiments, by physically decoupling sensing layers from associated electrode contacts and electrical interconnects, the methodology allows for the electrode contacts and electrical interconnects to be reused (as they do not come into direct contact with a fluid sample). In the methodology, a sensing layer is formed on a transferable, conductive, adhesive substrate which can be attached onto an electrode contact. After a sensing operation, the sensing layer can be detached from the electrode contact, and another fresh/uncontaminated sensing layer can be attached onto the electrode contact. With the methodology, the disposable part is a sensing layer while an electrode contact can be reused. Furthermore, in a sensor fabrication methodology of some embodiments, an activity of a capture probe/enzyme is preserved through applying freeze-drying (lyophilization) to facilitate extended operation in uncontrolled environments (e.g., on-body wearable analysis). Demonstration of the methodology is performed in the context of enzymatic sensors such as glucose and lactate sensors. For example, to realize a lactate sensor, a layer of gold and a layer of Prussian blue are respectively evaporated and electrodeposited on a conductive tape to promote electron transfer. Then, a mixture of chitosan/carbon nanotubes/lactate oxidase in a liquid medium is deposited via drop casting or spin coating as a sensing layer. After this chemical modification, a resulting lactate sensor is transferred to a freeze-drier. Through a freeze-drying operation, the encapsulated lactate oxidase-coated sensor can remain in a stable, solid form when not in use at about room temperature.

The methodology significantly lowers a development/production cost of biofluid analysis platforms through realizing a sensing interface which allows for reusing of electrode contacts and electrical interconnects (and discarding an electrochemical sensing layer after use). Therefore, the methodology provides a cost-effective solution for wearable and mobile biofluid analysis platforms which specify frequent sample analysis using a fresh/uncontaminated sensing interface, and can pave a path towards rendering sweat-based sensors scalable. By using sweat sensing for physiological monitoring, an improved diagnostic platform is provided, with real-time information sensing and transmission capabilities, and which is scalable and can be used to facilitate large-scale clinical investigations, remote patient monitoring, disease prevention/management, pharmaceutical monitoring, and patient performance monitoring.

In some embodiments, a disposable sensor for biofluid analysis includes: (1) a conductive film having a first major surface and a second major surface opposite to the first major surface; (2) a sensing layer disposed on the first major surface of the conductive film; and (3) an adhesive layer disposed on the second major surface of the conductive film.

In some embodiments, a method for biofluid analysis includes: (1) providing the disposable sensor of any of the foregoing embodiments; (2) attaching the disposable sensor onto an electrode contact of a wearable device; (3) exposing the disposable sensor to a biofluid during a sensing operation; and (4) detaching the disposable sensor from the electrode contact subsequent to the sensing operation.

In some embodiments, a method of forming a disposable sensor for biofluid analysis includes: (1) providing a coating composition including an enzyme; (2) applying the coating composition on a conductive film to form a coating on the conductive film; and (3) freeze-drying the coating to form a sensing layer on the conductive film.

In some embodiments, a disposable sensor array for biofluid analysis includes: (1) a conductive film having a first major surface and a second major surface opposite to the first major surface; (2) a first sensor disposed on the first major surface of the conductive film; (3) a second sensor disposed on the first major surface of the conductive film; and (4) an adhesive layer disposed on the second major surface of the conductive film.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1: Schematic diagram of a representative enzymatic sensor, where a disposable working electrode (WE) can be taped onto a corresponding electrode contact of a backside of a smartwatch (as an electronic reader). The placement of a reference electrode follows a same procedure.

FIG. 2: Calibration curves of glucose (a) and lactate (b) sensors (characterized in phosphate-buffered saline), demonstrating a high degree of linearity of sensor responses.

FIG. 3: Interference evaluation of glucose sensor response: steady state (a) and corresponding amperometric response (b). Results of corresponding interference evaluation of a lactate sensor are shown in (c) and (d).

FIG. 4: Sweat glucose levels of three subjects during about 12 h fasting state and about 30 min after about 30 g of glucose intake.

FIG. 5: On-body sweat lactate measurement during physical exercise (stationary cycling with three different intensities). Measured readings are low-pass filtered at about 0.1 Hz in digital domain. The upper curve indicates the heart rate profile, measured by a commercial heart rate sensor (left axis) and the lower curve shows the sweat lactate concentration profile (right axis). The exercise intensity was increased at two stages (about 700 s and about 900 s after beginning the exercise), which was immediately followed by an increase in the measured heart rate. The sweat secretion initiated at about 800 s after beginning the exercise and the sweat lactate level was elevated in response to the second increase in exercise intensity.

FIG. 6: Schematic of a wearable device.

FIG. 7: Schematic of a disposable working electrode of a sensor.

FIG. 8: Schematic of a sensor array patterned on a conductive substrate.

### DETAILED DESCRIPTION

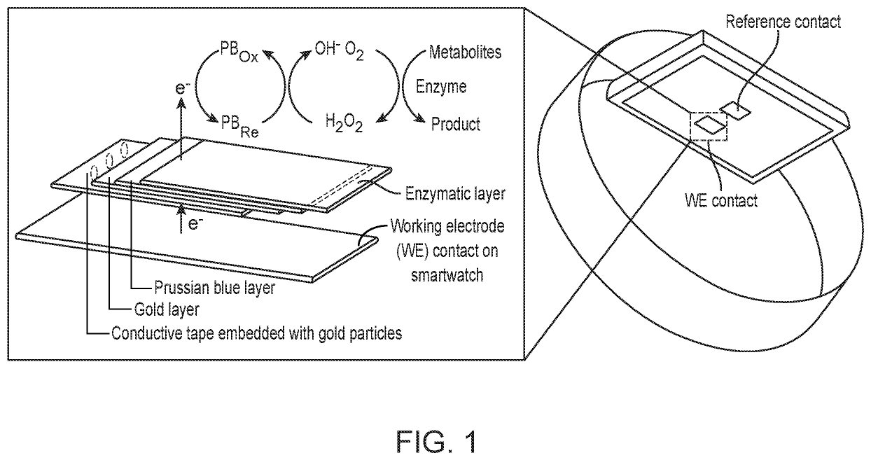

The exponential growth in Internet of Things (IoT) devices and wearable sensing technologies have created an unprecedented opportunity for personalized medicine, through real-time biomonitoring of individuals and allowing actionable feedback. Comparison IoT devices and wearable sensors are capable of tracking physical activities and vital signs but lack capability to access molecular-level biomarker information to provide insight into the body's dynamic chemistry. Sweat-based wearable biomonitoring has emerged as a candidate to merge this gap. Sweat is a rich source of biomarkers that can be retrieved unobtrusively. Sweat analysis platforms have demonstrated the in-situ measurement of sweat analytes in wearable formats. However, the lack of suitable sensor fabrication/integration schemes continues to impede the incorporation of sensors into wearable technologies to scale for population-level adoption. Specifically, proposed platforms are composed of physically-decoupled sensor arrays and readout circuit board modules and rely on two-dimensional (2D) electrical connections (on a same plane as a sensing interface) and cables to relay a transduced signal. Therefore, the platforms are spatially inefficient and their integration into wearable technologies is non-trivial. To overcome these bottlenecks, here, some embodiments are directed to a sensor fabrication/integration methodology, which allows for seamless and compact integration of disposable electrochemical sensors with permanent readout electronics. As shown in FIG. 1, in the methodology, an electrochemical sensing layer is formed on a vertically-conductive, adhesive substrate that can be attached onto/detached from electrode contacts of a wearable electronic reader (or other wearable device). As a demonstration, the methodology is applied to form enzymatic glucose and lactate sensors, and their functionalities are validated by performing human sweat sample analysis.

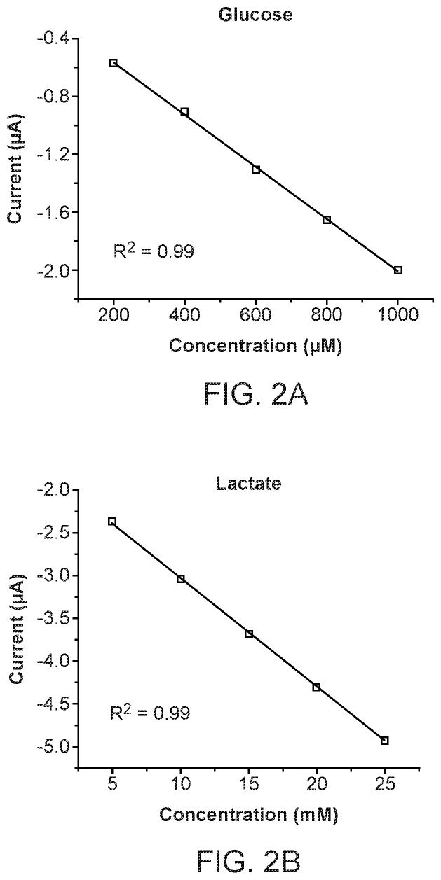

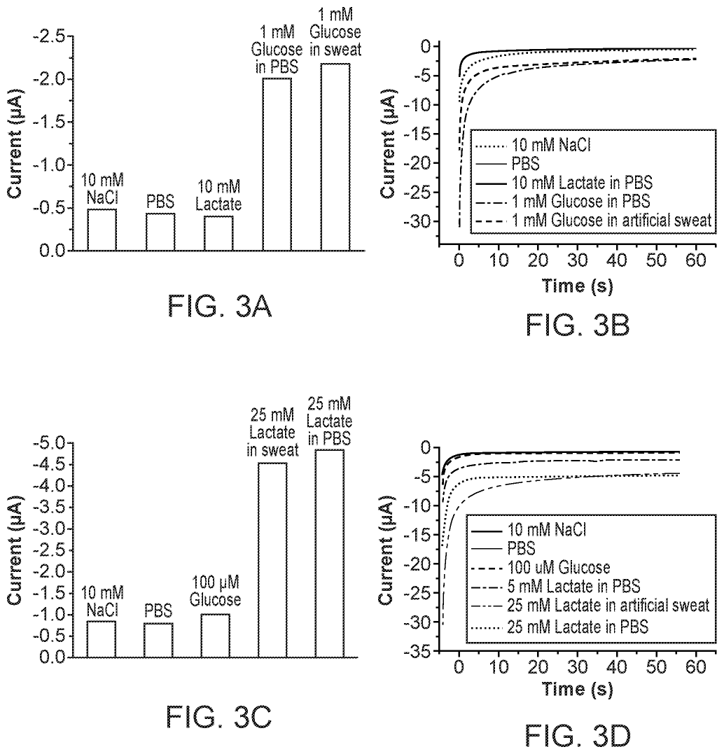

To form the sensing layer, gold is first evaporated on a z-axis electrically conductive, adhesive tape (which incorporates electrically conductive fillers in the form of gold particles, embedded in its structure, for electron transfer in a vertical direction). Then, a resulting gold-coated surface is functionalized with glucose/lactate oxidase enzymes entrapped in chitosan films. These sensing interfaces effectively output electrical current in correlation to a concentration of target analytes. Because of the sensor structure's z-direction electron transfer property, and stable adhesion to electrode contacts of printed circuit boards or other substrates (including gold and copper), the electrochemically-functionalized tape can be vertically integrated into electronic devices (e.g., a smartwatch). FIG. 2 illustrates calibration curves for the glucose and lactate sensors, demonstrating the corresponding sensors' highly linear responses (R²=about 0.99) within physiologically relevant ranges of concentrations. Validation is performed of the selectivity of the sensors, by evaluating the effect of non-target analytes (present in sweat) on sensor responses. As can be seen in FIG. 3, output current levels of the sensors due to interfering analytes are negligible as compared to those generated in response to target analytes.

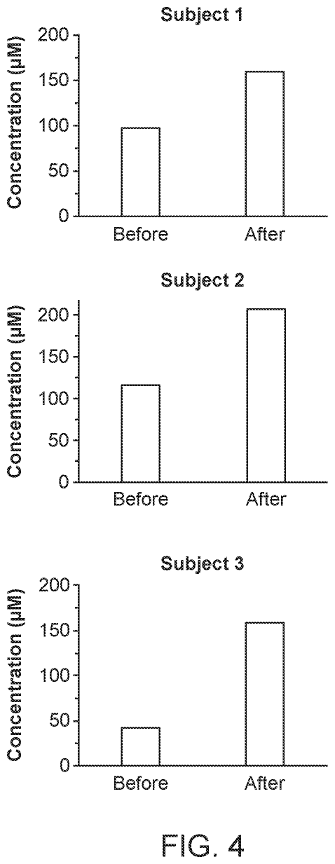

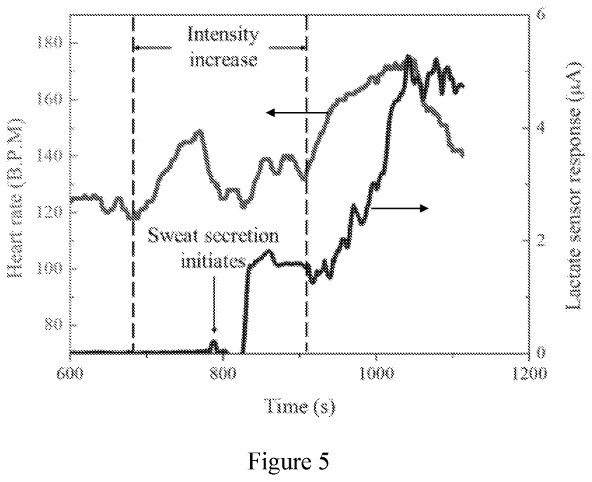

To validate the glucose sensor functionality, iontophoretically-stimulated sweat samples are collected from three subjects during about 12 h fasting and about 0.5 h after glucose intake (about 30 g glucose). As shown in FIG. 4, the sweat glucose level is noticeably increased in all three subjects. Additionally, the lactate sensor is integrated into a smartwatch to perform real-time sweat analysis during a graded-load cycling exercise (FIG. 5). In this evaluation, the exercise intensity was increased at two stages (about 700 s and about 900 s after beginning the exercise). The sweat secretion initiated at about 800 s after beginning the exercise. The wirelessly transmitted sweat lactate information demonstrated that the readily stabilized sweat lactate concentration elevated in response to the second increase in the exercise intensity level.

The scalable sensor fabrication and seamless integration methodology pave the way for incorporation of sweat sensors in wearable technologies for general population health monitoring.

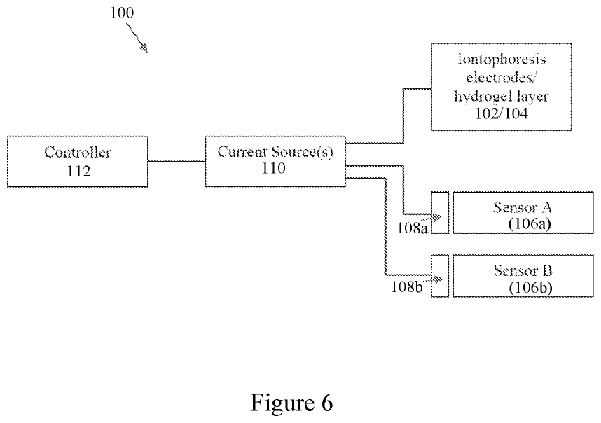

FIG. 6 is a schematic illustration of a wearable device 100 for sweat analysis according to some embodiments. The wearable device 100 includes a pair of iontophoresis electrodes 102/hydrogel layer 104 for sweat induction, and an array of sweat analyte sensors A and B. The hydrogel layer 104 is adjacent to the iontophoresis electrodes 102, and the iontophoresis electrodes 102 are configured to interface a skin with the hydrogel layer 104 in between. The hydrogel layer 104 includes a secretory agonist (e.g., a cholinergic sweat gland secretory stimulating compound, such as pilocarpine), which is released when an electrical current is applied to the iontophoresis electrodes 102. Each of the sensors A and B includes a working electrode 106a or 106b and a reference electrode. The electrodes included in the sensors A and B are disposable, and are removably attached via respective electrode contacts 108a and 108b to a remainder of the wearable device 100. The sensors A and B are configured to sense respective and different analytes, by generating sensing signals responsive to presence or levels of such analytes in induced sweat. For example, analytes can be selected from metabolites, electrolytes, proteins, and heavy metals. For example, the sensors A and B can be different sensors selected from a glucose sensor including an enzyme in a sensing layer (e.g., glucose oxidase), a lactate sensor including an enzyme in a sensing layer (e.g., lactate oxidase), a Na⁺ sensor, a Cl⁻ sensor, and Ca²⁺ sensor. Although the two sensors A and B are illustrated in FIG. 6, in general, one or more sensors can be included in the wearable device 100.

As shown in FIG. 6, the wearable device 100 also includes a set of current sources 110, which are connected to the iontophoresis electrodes 102 to activate sweat induction, and are connected to the sensors A and B to activate measurements of analyte concentrations. In some embodiments, multiple ones of the current sources 110 are included, and are connected to respective ones of the iontophoresis electrodes 102 and the sensors A and B. A controller 112 (e.g., including a processor and an associated memory storing processor-executable instructions) is also included in the wearable device 100, and is configured to control operation of various components of the wearable device 100. In particular, the controller 112 is configured to direct operation of the iontophoresis electrodes 102 and the sensors A and B, through control of the current sources 110. In addition, the controller 112 is configured to identify a presence of target analytes and derive concentration measurements of the target analytes. Although not shown, a wireless transceiver also can be included to allow wireless communication between the wearable device 100 and an external electronic device, such as a portable electronic device or a remote computing device.

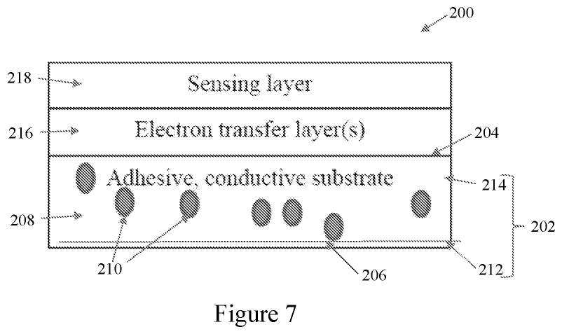

FIG. 7 is a schematic illustration of a disposable working electrode 200 according to some embodiments. The working electrode 200 includes a conductive substrate 202 which includes a conductive film 214 having a top major surface 204 and a bottom major surface 206. The conductive film 214 has anisotropic electrical conductivity, such that electrical conductivity is higher or preferential along one or more directions. In some embodiments, the conductive film 214 has a higher electrical conductivity along a direction extending between the top major surface 204 and the bottom major surface 206, and substantially perpendicular to the top major surface 204 or the bottom major surface 206, relative to its electrical conductivity along a direction substantially parallel to the top major surface 204 or the bottom major surface 206. The conductive film 214 can be formed of, or can include, a polymeric material 208 and electrically conductive fillers 210 (e.g., metallic particles) dispersed or embedded within the polymeric material 208 to impart anisotropic electrical conductivity. The conductive substrate 202 also includes an adhesive layer 212 formed of, or including, an adhesive material disposed on the bottom major surface 206 of the conductive film 214, thereby allowing the conductive substrate 202 to be attached onto and detached from an electrode contact.

As shown in FIG. 7, the working electrode 200 also includes a set of charge transfer layers 216 disposed on the conductive substrate 202, and, in particular, disposed on the top major surface 204 of the conductive film 214. The charge transfer layers 216 facilitate the transfer of electrical charges (e.g., electrons) between a sensing layer 218, which is disposed on the charge transfer layers 216, and the underlying conductive substrate 202. In some embodiments, the charge transfer layers 216 include a metallic layer, such as formed of, or including, gold or another metal, and an electrochemically active layer, such as formed of, or including, Prussian blue or another electrochemically active species capable of undergoing reduction and oxidation. The sensing layer 218 includes capture probes or an enzyme. In the case of an enzyme, the sensing layer 218 can include a biocompatible material, such as a biocompatible polymeric material, in which the enzyme is dispersed or embedded, optionally along with electrically conductive fillers (e.g., conductive carbonaceous particles). During fabrication of the working electrode 200, a coating composition including a mixture of the enzyme, the biocompatible material, and the conductive fillers in a liquid medium can be deposited or otherwise applied to form a coating on the conductive substrate 202, followed by freeze-drying to remove the liquid medium and impart stability to the resulting sensing layer 218. A reference electrode can be similarly configured as explained for the working electrode 200, with the omission of a sensing layer.

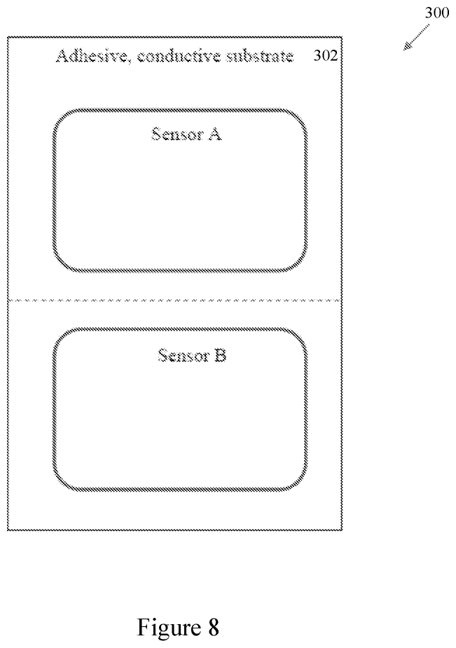

FIG. 8 is a schematic illustration of a sensor array 300 according to some embodiments. The sensor array 300 includes multiple sensors A and B patterned on a common conductive substrate 302. Each of the sensors A and B includes a working electrode, which includes a sensing layer and a set of charge transfer layers as explained in connection with FIG. 7. As shown in FIG. 8, the sensors A and B are formed as discrete, spatially segregated coating regions on respective areas of the conductive substrate 302, and, during use, the sensors A and B can be separated from one another, such as by cutting or subdividing along a dashed line. Anisotropic electrical conductivity of the conductive substrate 302 also allows the sensors A and B to operate even without cutting or subdividing, by preferentially conducting charges between the sensors A and B and their respective electrode contacts, while impeding against signal cross-coupling. The sensors A and B are configured to sense respective and different analytes. Other embodiments are contemplated, such in which the sensors A and B are configured to sense a same analyte, and in which the coating regions merge together as a contiguous coating on the conductive substrate 302.

### Example Embodiments

The following are example embodiments of this disclosure.

First Aspect

In some embodiments, a disposable sensor for biofluid analysis includes: (1) a conductive film having a first major surface and a second major surface opposite to the first major surface; (2) a sensing layer disposed on the first major surface of the conductive film; and (3) an adhesive layer disposed on the second major surface of the conductive film.

In any of the foregoing embodiments, the conductive film has an anisotropic electrical conductivity. In some embodiments, the conductive film has a higher electrical conductivity along a direction extending between the first major surface and the second major surface, relative to an electrical conductivity along a direction parallel to the first major surface or the second major surface.

In any of the foregoing embodiments, the conductive film includes conductive fillers dispersed therein. In some embodiments, the conductive fillers include metallic particles.

In any of the foregoing embodiments, the disposable sensor further includes a set of charge transfer layers disposed between the sensing layer and the conductive film. In some embodiments, the set of charge transfer layers includes a metallic layer. In some embodiments, the set of charge transfer layers includes an electrochemically active layer.

In any of the foregoing embodiments, the sensing layer includes an enzyme. In some embodiments, the sensing layer includes a polymeric material, and the enzyme is dispersed within the polymeric material.

Second Aspect

In some embodiments, a method for biofluid analysis includes: (1) providing the disposable sensor of any of the foregoing embodiments of the first aspect; (2) attaching the disposable sensor onto an electrode contact of a wearable device; (3) exposing the disposable sensor to a biofluid during a sensing operation; and (4) detaching the disposable sensor from the electrode contact subsequent to the sensing operation.

Third Aspect

In some embodiments, a method of forming a disposable sensor for biofluid analysis includes: (1) providing a coating composition including an enzyme; (2) applying the coating composition on a conductive film to form a coating on the conductive film; and (3) freeze-drying the coating to form a sensing layer on the conductive film.

In any of the foregoing embodiments, the conductive film has an anisotropic electrical conductivity.

In any of the foregoing embodiments, the coating composition is applied on a first major surface of the conductive film, and an adhesive layer is disposed on a second major surface of the conductive film that is opposite to the first major surface.

Fourth Aspect

In some embodiments, a disposable sensor array for biofluid analysis includes: (1) a conductive film having a first major surface and a second major surface opposite to the first major surface; (2) a first sensor disposed on the first major surface of the conductive film; (3) a second sensor disposed on the first major surface of the conductive film; and (4) an adhesive layer disposed on the second major surface of the conductive film.

In any of the foregoing embodiments, the conductive film has an anisotropic electrical conductivity.

In any of the foregoing embodiments, the conductive film includes conductive fillers dispersed therein.

In any of the foregoing embodiments, the first sensor and the second sensor are spatially segregated from one another on the first major surface of the conductive film.

In any of the foregoing embodiments, the first sensor includes a first sensing layer and a first set of charge transfer layers disposed between the first sensing layer and the conductive film, and the second sensor includes a second sensing layer and a second set of charge transfer layers disposed between the second sensing layer and the conductive film. In some embodiments, the first sensing layer includes a first enzyme, and the second sensing layer includes a second enzyme. In some embodiments, the first enzyme and the second enzyme are different.

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common characteristics.

As used herein, the terms “connect,” “connected,” and “connection” refer to an operational coupling or linking. Connected objects can be directly coupled to one another or can be indirectly coupled to one another, such as via one or more other objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, a first numerical value can be “substantially” or “about” the same as a second numerical value if the first numerical value is within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, substantially parallel can refer to a range of angular variation relative to 0° of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°. For example, substantially perpendicular can refer to a range of angular variation relative to 90° of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

In the description of some embodiments, a component provided “on” or “over” another component can encompass cases where the former component is directly on (e.g., in physical contact with) the latter component, as well as cases where one or more intervening components are located between the former component and the latter component.

Additionally, concentrations, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

Some embodiments of this disclosure relate to a non-transitory computer-readable storage medium having computer code or instructions thereon for performing various processor-implemented operations. The term “computer-readable storage medium” is used to include any medium that is capable of storing or encoding a sequence of instructions or computer code for performing the operations, methodologies, and techniques described herein. The media and computer code may be those specially designed and constructed for the purposes of the embodiments of the disclosure, or they may be of the kind available to those having skill in the computer software arts. Examples of computer-readable storage media include volatile and non-volatile memory for storing information. Examples of memory include semiconductor memory devices such as erasable programmable read-only memory (EPROM), electrically erasable programmable read-only memory (EEPROM), random-access memory (RAM), and flash memory devices, discs such as internal hard drives, removable hard drives, magneto-optical, compact disc (CD), digital versatile disc (DVD), and Blu-ray discs, memory sticks, and the like. Examples of computer code include machine code, such as produced by a compiler, and files containing higher-level code that are executed by a processor using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computing device via a transmission channel. Another embodiment of the disclosure may be implemented in hardwired circuitry in place of, or in combination with, processor-executable software instructions.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

1. A disposable sensor for biofluid analysis, comprising:
a conductive film having a first major surface and a second major surface opposite to the first major surface;
a sensing layer disposed on the first major surface of the conductive film; and
an adhesive layer disposed on the second major surface of the conductive film.

2. The disposable sensor of claim 1, wherein the conductive film has an anisotropic electrical conductivity.

3. The disposable sensor of claim 2, wherein the conductive film has a higher electrical conductivity along a direction extending between the first major surface and the second major surface, relative to an electrical conductivity along a direction parallel to the first major surface or the second major surface.

4. The disposable sensor of claim 1, wherein the conductive film includes conductive fillers dispersed therein.

5. The disposable sensor of claim 4, wherein the conductive fillers include metallic particles.

6. The disposable sensor of claim 1, further comprising a set of charge transfer layers disposed between the sensing layer and the conductive film.

7. The disposable sensor of claim 6, wherein the set of charge transfer layers includes a metallic layer.

8. The disposable sensor of claim 6, wherein the set of charge transfer layers includes an electrochemically active layer.

9. The disposable sensor of claim 1, wherein the sensing layer includes an enzyme.

10. The disposable sensor of claim 9, wherein the sensing layer includes a polymeric material, and the enzyme is dispersed within the polymeric material.

11. A disposable sensor array for biofluid analysis, comprising:
a conductive film having a first major surface and a second major surface opposite to the first major surface;
a first sensor disposed on the first major surface of the conductive film;
a second sensor disposed on the first major surface of the conductive film; and
an adhesive layer disposed on the second major surface of the conductive film.

12. The disposable sensor array of claim 11, wherein the conductive film has an anisotropic electrical conductivity.

13. The disposable sensor array of claim 11, wherein the conductive film includes conductive fillers dispersed therein.

14. The disposable sensor array of claim 11, wherein the first sensor and the second sensor are spatially segregated from one another on the first major surface of the conductive film.

15. The disposable sensor array of claim 11, wherein:
the first sensor includes a first sensing layer and a first set of charge transfer layers disposed between the first sensing layer and the conductive film; and
the second sensor includes a second sensing layer and a second set of charge transfer layers disposed between the second sensing layer and the conductive film.

16. A method for biofluid analysis, comprising:
providing the disposable sensor of claim 1;
attaching the disposable sensor onto an electrode contact of a wearable device;
exposing the disposable sensor to a biofluid during a sensing operation; and
detaching the disposable sensor from the electrode contact subsequent to the sensing operation.

17. A method of forming a disposable sensor for biofluid analysis, comprising:
providing a coating composition including an enzyme;
applying the coating composition on a conductive film to form a coating on the conductive film; and
freeze-drying the coating to form a sensing layer on the conductive film.

18. The method of claim 17, wherein the conductive film has an anisotropic electrical conductivity.

19. The method of claim 17, wherein the coating composition is applied on a first major surface of the conductive film, and an adhesive layer is disposed on a second major surface of the conductive film that is opposite to the first major surface.
